(19) United States
(12) Patent Application Publication
(10) Pub. No.: US 2007/0117103 A1
(43) Pub. Date: May 24, 2007

(54) Nucleotide analogs

(76) Inventor: Philip R. Buzby, Brockton, MA (US)

Correspondence Address: GOODWIN PROCTER LLP;PATENT ADMINISTRATOR, EXCHANGE PLACE, BOSTON, MA, 02109-2881, US

(21) Appl. No.: 11/286,626

(22) Filed: Nov. 22, 2005

(51) Int. Cl. C12Q 1/68; C07H 21/04

(52) U.S. Cl. 435/6; 536/25.32

(57) ABSTRACT

The invention provides nucleotide analogs for use in sequencing nucleic acid molecules.

### FIELD OF THE INVENTION

The invention relates to nucleotide analogs and methods for sequencing a nucleic acid using the nucleotide analogs.

### BACKGROUND

There have been proposals to develop new sequencing technologies based on single-molecule measurements. For example, sequencing strategies have been proposed that are based upon observing the interaction of particular proteins with DNA or by using ultra high resolution scanned probe microscopy. See, e.g., Rigler, et al., J. Biotechnol., 86(3):161 (2001); Goodwin, P. M., et al., Nucleosides & Nucleotides, 16(5-6):543-550 (1997); Howorka, S., et al., Nature Biotechnol., 19(7):636-639 (2001); Meller, A., et al., Proc. Nat'l. Acad. Sci., 97(3):1079-1084 (2000); Driscoll, R. J., et al., Nature, 346(6281):294-296 (1990).

Recently, a sequencing-by-synthesis methodology has been proposed that resulted in sequence determination, but not with consecutive base incorporation. See, Braslavsky, et al., Proc. Nat'l Acad. Sci., 100: 3960-3964 (2003). An impediment to base-over-base sequencing has been the use of bulky flourophores that can sterically hinder sequential base incorporation. Even when the label is cleaved, some fluorescently-labeled nucleotides sterically hinder subsequent base incorporation due to the residue of the linker left behind after cleavage.

A need therefore exists for nucleotide analogs having reduced steric hindrance, thereby allowing the polymerase to produce greater read-length from each template.

### SUMMARY OF THE INVENTION

The present invention provides nucleotide analogs and methods of using nucleotide analogs in sequencing. A nucleotide analog of the invention comprises a cleavable linker between the base portion of the nucleotide and the label such that, upon cleavage, the analog does not substantially hinder subsequent nucleotide (or nucleotide analog) incorporation. Prior to cleavage, analogs of the invention allow only limited base addition in any given cycle of template-dependent nucleotide incorporation.

In a preferred embodiment, a nucleotide analog of the invention is a nucleotide triphosphate comprising an optically-detectable label attached to the nitrogenous base portion of the nucleotide via a cleavable linker. The cleavable site in the linker is sufficiently close to the base such that, when cleaved, it leaves a “stub” that does not interfere with subsequent base incorporation. A preferred linker is between about 0 and about 10 atoms in length, preferably between about 1 and about 7 atoms in length. Examples of preferred linkers are provided herein.

After cleavage, the remaining linker portion is ideally between about 1 and about 5 atoms in length. In certain embodiments, the linker may be capped after cleavage to render it unreactive. In a highly-preferred embodiment, the linker is cleaved back to the original base structure. However, in any case, cleavage should leave as little of the linker as possible attached to the base portion of the nucleotide.

Cleavage may be accomplished via any appropriate method. For example, a cleavage site may be chemically cleavable, photolytically cleavable, or mechanically cleavable (i.e., by shaking). Specific examples are provided below. A preferred cleavage site is a disulfide linker, which can easily be positioned in the linker in order to effect the purposes of the invention.

Any detectable label can be used in practice of the invention. Optically-detectable labels, and particularly fluorescent labels, are highly preferred. The base is selected from the group consisting of a purine, a pyrimidine and derivatives. Analogs of the invention may be further modified by inclusion of a blocking group at the 3′ hydroxyl position on the sugar moiety of the nucleotide. For example, a preferred analog comprises a phosphate group in place of the hydroxyl group in the 3′ position of the nucleotide sugar.

Certain nucleotide analogs of the invention also include a sulfur in place of a non-bridging oxygen at the a phosphate of the nucleotide triphosphate. The presence of a sulfur in place of a non-bridging oxygen of the a phosphate group causes the nucleotide analog and polynucleotide comprising one or more of such nucleotide analogs to be resistant to nuclease activity, particularly nuclease activity that may be associated with enzymes used to remove the optional phosphate group in place of the hydroxyl group at the 3′ position of the nucleotide sugar.

In general, methods of using nucleotide analogs of the invention comprise exposing a target nucleic acid/primer duplex to one or more nucleotide analogs and a polymerase under conditions suitable to extend the primer in a template dependent manner. Any appropriate polymerase can be used according to the invention. For example, in one embodiment, a Klenow fragment with reduced exonuclease activity is used to extend the primer in a template-dependent manner. Generally, the primer is, or is made to be, sufficiently complementary to at least a portion of the target nucleic acid to hybridize to the target nucleic acid and allow template-dependent nucleotide polymerization. The primer is extended by one or more bases.

In one embodiment, a labeled nucleotide analog having a linker between about 5 carbon atoms and about 1 carbon atom between a cleavable site and the base portion of the nucleotide is incorporated into a primer portion of a nucleic acid duplex comprising a template to be sequenced hybridized to the primer. The incorporated labeled nucleotide is identified and the cleavable bond is cleaved. The incorporating, identifying, and cleaving steps are repeated at least one time and a sequence of the target nucleic acid/primer duplex is determined based upon the order of the incorporation of the labeled nucleotides. Optionally, the cleaved bond is capped (for example, with an alkylating agent), rendering it unreactive. Alkylating agents, such as iodoacetamide, are used to cap the cleaved bond.

In another embodiment, a labeled nucleotide analog having a linker greater than 5 carbon atoms between a base and a label is incorporated in the target nucleic acid. The incorporated labeled nucleotide is identified. The label and at least a portion of the linker are removed such that a remaining portion of the linker has 7 or fewer atoms. The incorporating, identifying, and removing steps are repeated at least one time and a sequence of the target nucleic acid/primer duplex is determined based upon the order of the incorporation of the labeled nucleotides. Optionally, the remaining portion of the linker is capped, rendering it unreactive.

In one embodiment, a labeled nucleotide analog having a linker between about 5 carbon atoms and about 1 carbon atom is incorporated in the target nucleic acid. The incorporated labeled nucleotide is identified by the label. The label is removed, the incorporating, identifying, and removing steps are repeated at least one time, and a sequence of the target nucleic acid/primer duplex is determined based upon the order of the incorporation of the labeled nucleotides.

In single molecule sequencing, a template nucleic acid molecule/primer duplex is immobilized on a surface such that nucleotides (or nucleotide analogs) added to the immobilized primer are individually optically resolvable. Either the primer, template and/or nucleotide analogs can be detectably labeled such that the position of the duplex is individually optically resolvable. The primer can be attached to the solid support, thereby immobilizing the hybridized template nucleic acid molecule, or the template can be attached to the solid support thereby immobilizing the hybridized primer. The primer and template can be hybridized to each other prior to or after attachment of either the template or the primer to the solid support. The detectable label preferably is optically-detectable, and most preferably is a fluorescent label. Any appropriate fluorescent label can be used according to the invention. For example, appropriate fluorescent labels include cyanine, rhodamine, fluorescien, coumarin, BODIPY, alexa, conjugated multi-dyes, or any combination of these.

Where an optional phosphate group is present in place of the hydroxyl in the 3′ position of the nucleotide sugar, the optional phosphate moiety is removed, preferably enzymatically, after incorporation in order to allow subsequent incorporations. The incorporated nucleotide analog can be detected before, during, or after removing the optional phosphate group.

The primer extension process can be repeated to identify additional nucleotide analogs in the template. The sequence of the template is determined by compiling the detected nucleotides, thereby determining the complimentary sequence of the target nucleic acid molecule.

In general, methods for facilitating the incorporation of a nucleotide analog in a primer include exposing a target nucleic acid/primer duplex to one or more nucleotide analogs of the present invention and a polymerase under conditions suitable to extend the primer in a template dependent manner. Generally, the primer is sufficiently complementary to at least a portion of the target nucleic acid to hybridize to the target nucleic acid and allow template-dependent nucleotide polymerization.

While the invention is exemplified herein with fluorescent labels, the invention is not so limited and can be practiced using nucleotides labeled with any detectable label, including chemiluminescent labels, luminescent labels, phosphorescent labels, fluorescence polarization labels, and charge labels.

A detailed description of the certain embodiments of the invention is provided below. Other embodiments of the invention are apparent upon review of the detailed description that follows.

### BRIEF DESCRIPTION OF THE DRAWINGS

FIG. 1 is a generic chemical structure of the nucleotide analog of the present invention having a linker attached to the base.

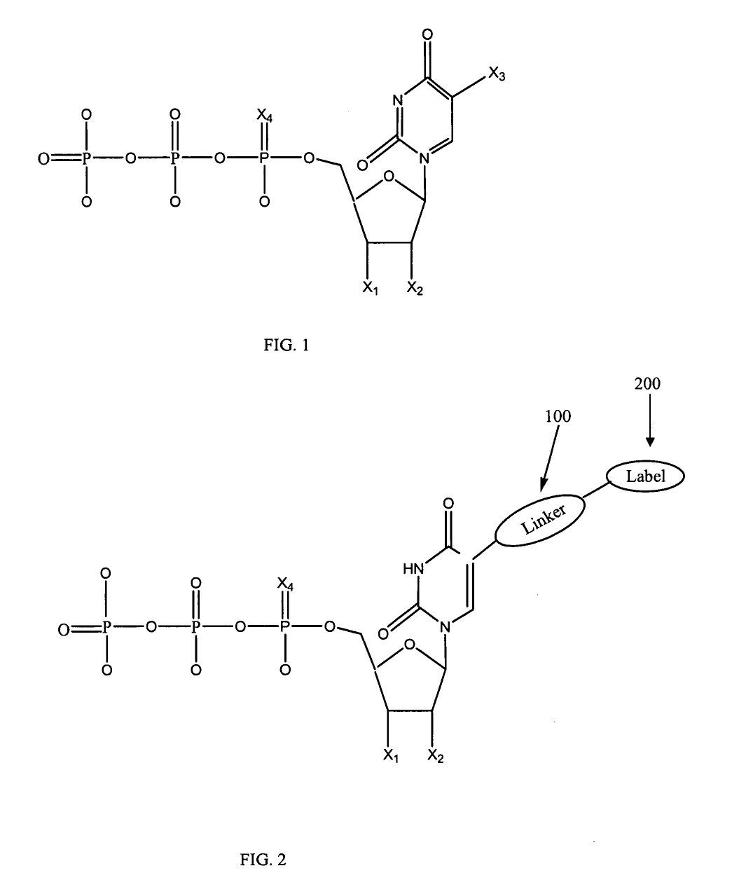

FIG. 2 is a generic chemical structure of the nucleotide analog of the present invention having a linker between the base and a label.

FIG. 3A shows a generic representation of nucleotide analogs of the present invention having a linker between the base and the label.

FIG. 3B shows exemplary chemical structures of nucleotide analogs of the present invention having a linker between the base and the label.

FIG. 4A shows a generic representation of nucleotide analogs of the present invention that undergo internal rearrangement of the linker between the base and a label.

FIG. 4B shows exemplary chemical structures of nucleotide analogs of the present invention that undergo internal rearrangement of the linker between the base and a label.

FIG. 5A shows a generic representation of nucleotide analogs of the present invention that undergo cleavage of the linker between the base and a label.

FIG. 5B shows exemplary chemical structures of nucleotide analogs of the present invention that undergo cleavage of the linker between the base and a label.

FIGS. 6A-6C show a method of making an exemplary nucleotide analog of the present invention. The nucleotide analog undergoes cleavage of the linker between the base and a label.

### DETAILED DESCRIPTION OF THE INVENTION

The invention relates generally to nucleotide analogs that, when used in sequencing reactions, allow extended base-over-base incorporation into a primer in a template-dependent sequencing reaction. Nucleotide analogs of the invention include nucleotide triphosphates having a linker between the base portion of the nucleotide and a detectable label, wherein the linker is cleavable to produce a residue that closely resembles the native (i.e., unlabeled) nucleotide. Analogs of the invention are useful in sequencing-by-synthesis reactions in which consecutive based are added to a primer in a template-dependent manner.

Nucleotide Analogs

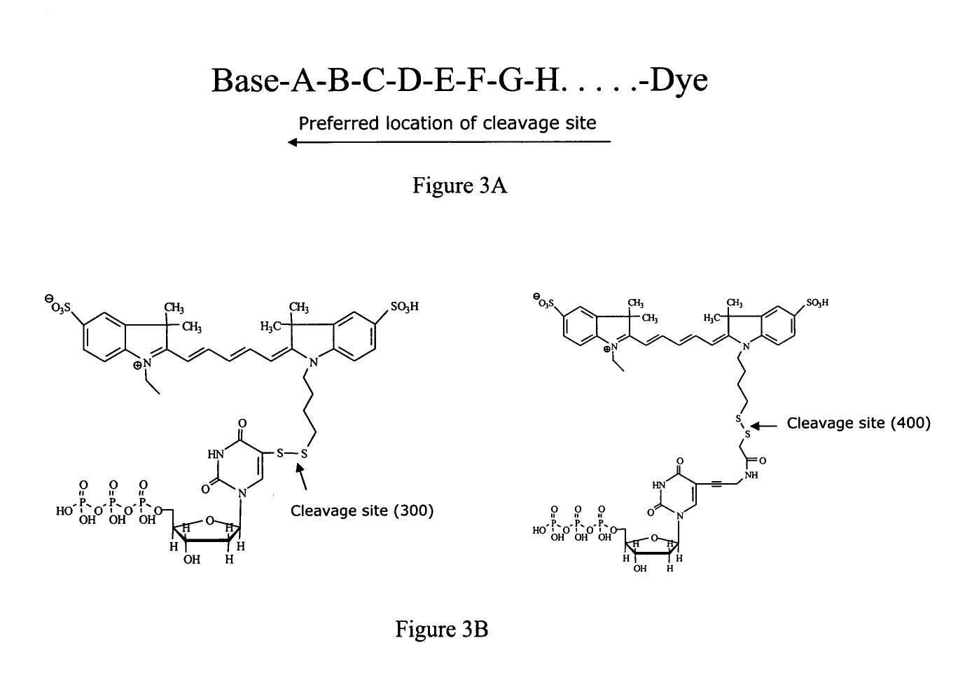

Preferred nucleotide analogs of the invention have the generalized structure: 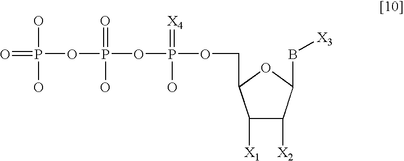
where X₁ can be OH or PO₄, X₂ can be H or OH, or X₄ can be O or S. Nucleotide analogs comprise a linker X₃ between the base portion (B) and a label (not shown). Preferably, the linker has between about 10 atoms and about 1 atom. A preferred linker features a cleavable bond. Referring now to FIG. 2, the linker 100 is attached to a label 200 such that the linker 100 is between the label 200 and a base. The linker preferably is a cleavable linker. The cleavable linker can be chemically cleavable, photo-cleavable, or mechanically cleavable. A preferred photochemically cleavable linker is an o-nitrobenzyl group or a derivative thereof. Suitable photochemically cleavable linkers are provided below. Chemically cleavable linkers can be cleaved under acidic, basic, oxidative, or reductive conditions. Examples of chemically cleavable linkers are provided below. In one embodiment, the chemically cleavable linker is a disulfide bond. The label can be an optically-detectable label, for example, a fluorescent label. Referring again to the above structure [10], the base B is a purine, deazapurine, pyrimidine, or derivative of any of the foregoing.

The base B can be, for example, adenine, cytosine, guanine, thymine, uracil, or hypoxanthine. The base B also can be, for example, naturally-occurring and synthetic derivatives of the preceding group, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine. Bases useful according to the invention permit a nucleotide that includes that base to be incorporated into a polynucleotide chain by a polymerase and will form base pairs with a base on an antiparallel nucleic acid strand. The term base pair encompasses not only the standard AT, AU or GC base pairs, but also base pairs formed between nucleotides and/or nucleotide analogs comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the nucleotide analog inosine and adenine, cytosine or uracil, where the two hydrogen bonds are formed.

The label preferably is a detectable label. In one embodiment, the label is an optically-detectable label such as a fluorescent label. The label can be selected from detectable labels including cyanine, rhodamine, fluorescien, coumarin, BODIPY, alexa, conjugated multi-dyes, or any combination of these. However, any appropriate detectable label can be used according to the invention, and are known to those skilled in the art.

As described above, the nucleotide analogs of the present invention also can include a moiety at the 3′ position of the nucleotide sugar that prevents further extension of the primer after the nucleotide analog has been added to the primer. In one embodiment, the 3′ position of the nucleotide sugar has a phosphate group in place of the standard hydroxyl group. In order to prevent or reduce degradation of the primer containing the nucleotide analog or degradation of the nucleotide analogs, the nucleotide analog can further comprise a non-bridging sulfur on the a phosphate group of the nucleotide.

Nucleic Acid Sequencing

The invention also includes methods for nucleic acid sequence determination using the nucleotide analogs described herein. The nucleotide analogs of the present invention are particularly suitable for use in single molecule sequencing techniques. Such techniques are described for example in U.S. patent application Ser. No. 10/831,214 filed April 2004; Ser. No. 10/852,028 filed May 24, 2004; Ser. No. 10/866,388 filed Jun. 10, 2005; Ser. No. 10/099,459 filed Mar. 12, 2002; and U.S. Published Application 2003/013880 published Jul. 24, 2003, the teachings of which are incorporated herein in their entireties. In general, methods for nucleic acid sequence determination comprise exposing a target nucleic acid (also referred to herein as template nucleic acid or template) to a primer that is complimentary to at least a portion of the target nucleic acid, under conditions suitable for hybridizing the primer to the target nucleic acid, forming a template/primer duplex.

Target nucleic acids include deoxyribonucleic acid (DNA) and/or ribonucleic acid (RNA). Target nucleic acid molecules can be obtained from any cellular material, obtained from an animal, plant, bacterium, virus, fungus, or any other cellular organism. Target nucleic acids may be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid molecules may also be isolated from cultured cells, such as a primary cell culture or a cell line. The cells from which target nucleic acids are obtained can be infected with a virus or other intracellular pathogen.

A sample can also be total RNA extracted from a biological specimen, a cDNA library, or genomic DNA. Nucleic acid typically is fragmented to produce suitable fragments for analysis. In one embodiment, nucleic acid from a biological sample is fragmented by sonication. Test samples can be obtained as described in U.S. Patent Application 2002/0190663 A1, published Oct. 9, 2003, the teachings of which are incorporated herein in their entirety. Generally, nucleic acid can be extracted from a biological sample by a variety of techniques such as those described by Maniatis, et al., Molecular Cloning: A Laboratory Manual, Cold Spring Harbor, N.Y., pp. 280-281 (1982). Generally, target nucleic acid molecules can be from about 5 bases to about 20 kb. Nucleic acid molecules may be single-stranded, double-stranded, or double-stranded with single-stranded regions (for example, stem- and loop-structures).

One or more nucleotide analogs as described herein and a polymerase are added to the template/primer duplex under conditions suitable for extending the primer in a template-dependant manner. The primer can be extended by one or more nucleotide analogs. The addition of the nucleotide analog to the primer results in the removal of the terminal two phosphate groups. The incorporated nucleotide analog is identified.

Any polymerase and/or polymerizing enzyme may be employed. A preferred polymerase is Klenow with reduced exonuclease activity. Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Komberg and Baker, W. H. Freeman, New York, N.Y. (1991). Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9°Nm™ DNA polymerase (New England Biolabs), Stoffel fragment, ThermoSequenase® (Amersham Pharmacia Biotech UK), Therminator™ (New England Biolabs), Thermotoga maritima (Tma) DNA polymerase (Diaz and Sabino, 1998 Braz J Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J Biol. Chem. 256:3112), and archaeal DP1I/DP2 DNA polymerase II (Cann et al., 1998, Proc Natl Acad. Sci. USA 95:14250>5).

Other DNA polymerases include, but are not limited to, ThermoSequenase®, 9°Nm™, Therminator™, Taq, Tne, Tma, Pfu, Tfl, Tth, Tli, Stoffel fragment, Vent™ and Deep Vent™ DNA polymerase, KOD DNA polymerase, Tgo, JDF-3, and mutants, variants and derivatives thereof. Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit Rev Biochem. 3:289-347(1975)).

Unincorporated nucleotide analog molecules are removed prior to or after detecting. Unincorporated nucleotide analog molecules can be removed by washing.

The template/primer duplex is then treated such that the label is removed or the linker is cleaved, partially removed and/or degraded. The steps of exposing template/primer duplex to one or more nucleotide analogs and polymerase, detecting incorporated nucleotides, and then treating to (1) remove and/or degrade the label, (2) remove and/or degrade the label and at least a portion of the linker or (3) cleave the linker can be repeated, thereby identifying additional bases in the template nucleic acid, the identified bases can be compiled, thereby determining the sequence of the target nucleic acid. In some embodiments, the remaining linker and label are not removed, for example, in the last round of primer extension.

One embodiment of a method for sequencing a nucleic acid template includes exposing a nucleic acid template to a primer capable of hybridizing to the template and a polymerase capable of catalyzing nucleotide addition to the primer. Incorporating a labeled nucleotide analog having a linker comprising between about 5 carbon atoms and about 1 carbon atom between a cleavable bond and a base. Identifying the incorporated labeled nucleotide. Once the labeled nucleotide is identified the cleavable bond is cleaved removing at least the label. The exposing, incorporating, identifying and cleaving steps are repeated at least once. The sequence of the template is determined based upon the order of incorporation of the labeled nucleotides.

In another embodiment, a method for sequencing a nucleic acid template includes exposing a nucleic acid template to a primer capable of hybridizing to the template and a polymerase capable of catalyzing nucleotide addition to the primer. The polymerase is, for example, Klenow with reduced exonuclease activity. Incorporating a labeled nucleotide analog having a linker greater than 5 carbon atoms between a base and a label. Identifying the incorporated labeled nucleotide. Once the labeled nucleotide is identified, the label and at least a portion of the linker are removed and the remaining portion of the linker has 5 or fewer carbon atoms. The exposing, incorporating, identifying, and removing steps are repeated at least once. The sequence of the template is determined based upon the order of incorporation of the labeled nucleotides.

In another embodiment, a method for sequencing a nucleic acid template includes exposing a nucleic acid template to a primer capable of hybridizing to the template and a polymerase capable of catalyzing nucleotide addition to the primer. Incorporating a labeled nucleotide analog having a linker comprising between about 5 carbon atoms and about 1 carbon atom. Identifying the incorporated labeled nucleotide. Once the labeled nucleotide is identified, the label is removed. The exposing, incorporating, identifying, and removing steps are repeated at least once. The sequence of the template is determined based upon the order of incorporation of the labeled nucleotides.

The above-described methods for sequencing a nucleic acid template can further include a step of capping the cleavable bond for example, after the bond has been cleaved. The methods for sequencing a nucleic acid template may employ a detectable label selected from, for example, cyanine, rhodamine, fluorescien, coumarin, BODIPY, alexa, conjugated multi-dyes or any combination of these. The template can be individually optically resolvable and is optionally attached to a surface.

In one embodiment, the cleavable linker X₃ is a photochemically cleavable linker and the linker is cleaved by exposing the extended primer to light of a suitable wavelength for a suitable duration of time to cleave the photochemically cleavable linker, thereby causing the removal of the label and at least a portion of the linker from the incorporated nucleotide analog.

In one embodiment, a cleavable linker and fluorescent dye is used (Scheme I). The cleavable linker is attached directly to the base B. In this scenario, once the nucleotide analog is added to the primer, the linker and the attached fluorescent dye are removed. Once the linker is cleaved, removing the fluorescent dye and a portion of the linker from the nucleotide analog, the analog closely resembles a native nucleotide. Further, any remaining portion of the linker present on the nucleotide analog will not interfere with subsequent addition of nucleotides to the primer. Although uridine is shown below as an example, all bases (A, U, C, G) and analogs are contemplated for use in the invention as described herein.

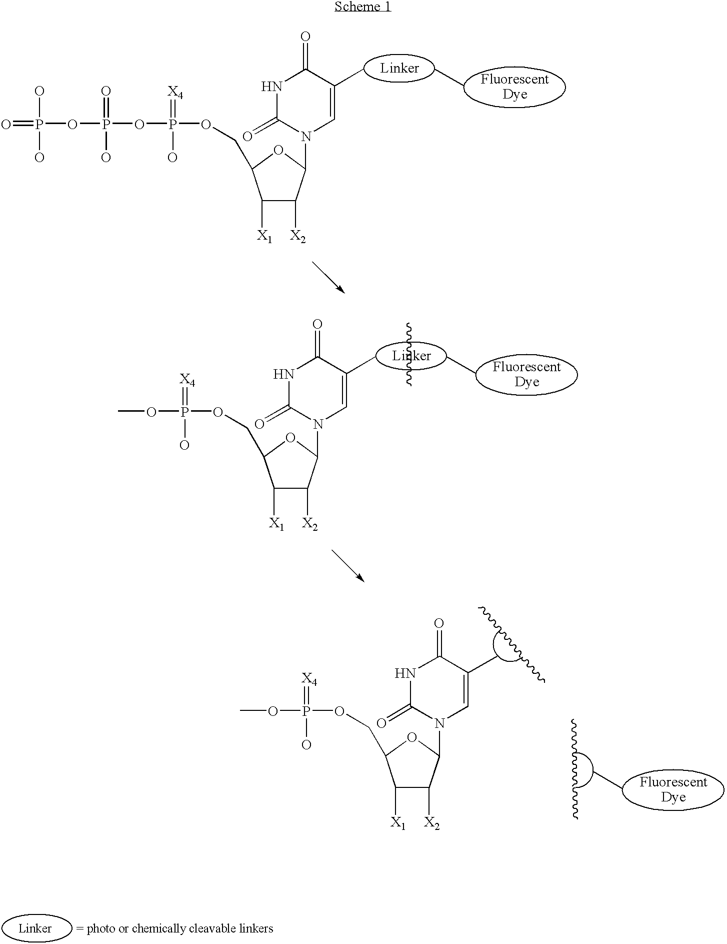

In one embodiment, according to Scheme 1, the linker features a cleavable bond, for example, a disulfide bond, which is located between about 5 carbon atoms and about 1 carbon atom from the uridine base. The disulfide bond can be cleaved using a reducing agent. Examples of additional cleavable bonds include the molecules provided directly below and their derivatives.

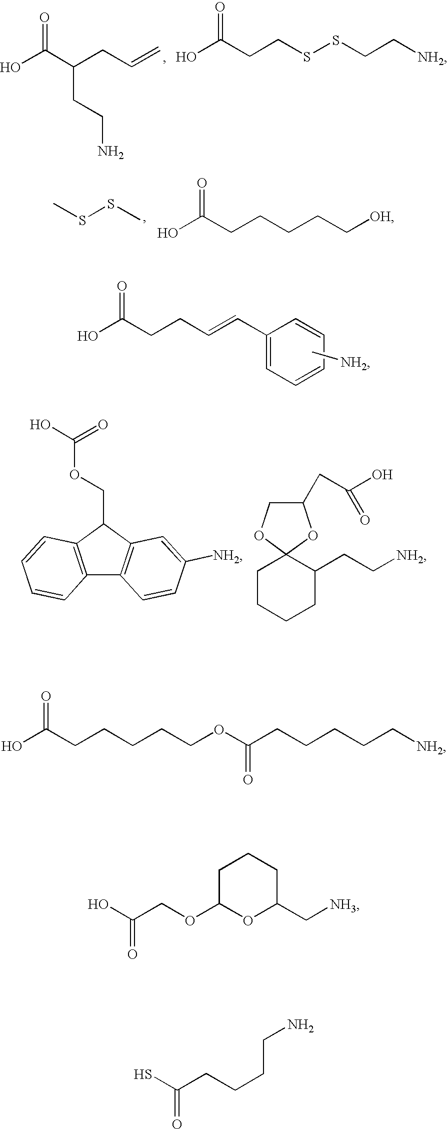

Examples of additional molecules suitable for use as linkers having photochemically cleavable bonds are provided below (16-19):

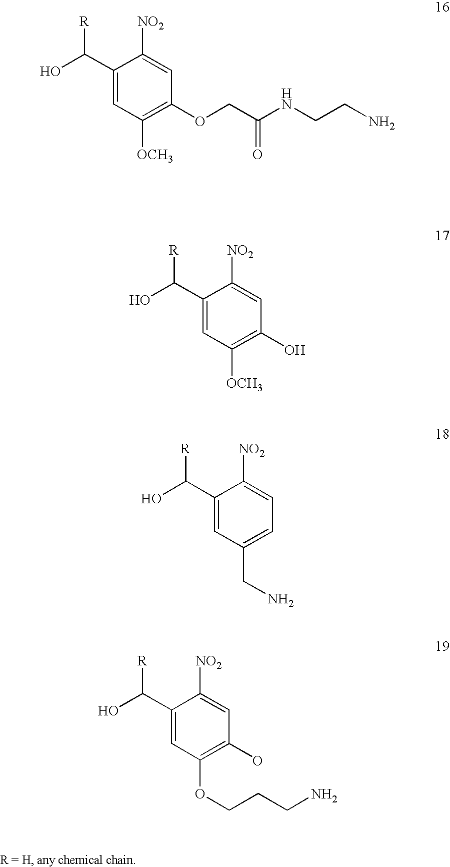

Linkers can be cleaved or degraded under acidic, basic, oxidative, or reductive conditions. In a preferred embodiment, chemical cleavage is accomplished using a reducing agent, such as TCEP (tris(2-carboxyethyl) phosphine hydrochloride), β-mercaptoethanol, or DTT (dithiothreitol). Optionally, the remaining portion of the linker is treated with an agent that renders it chemically unreactive. For example, if cleavage occurs at a disulfide bond, a sulfhydryl capping agent, such as iodoacetamide, is used.

In another embodiment, amino acid 25 or commercially available alcohol 24 can be linked to a fluorophore and then cleaved by either base or enzyme-promoted hydrolysis of the ester bond. Another base-labile linker is 26, which has similar reactivity to the FMOC (fluorenylmethoxycarbonyl) protecting group. Amino acid linkers 27 and 28 will allow for removal of the fluorescent dye under acidic conditions as the acetal moieties can be gently hydrolyzed. Alternatively, α-substituted pentenoic acid derivative 29 will promote the liberation of the fluorophore under oxidative iodolactonization conditions, while the disulfide functionality of 30 and 31 will provide a substrate suitable for reductive cleavage. Finally, linker diene 32 will allow for release of the fluorophore under aqueous ring closing metathesis conditions.

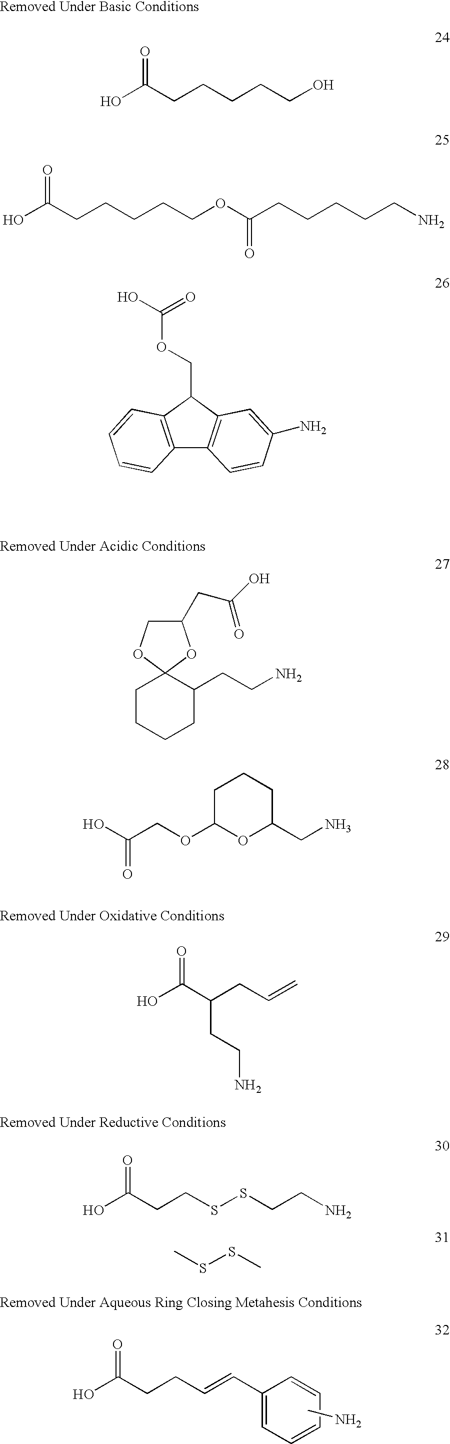

After addition of the nucleotide analog to the primer, the optional phosphate can be removed enzymatically. In one embodiment, the optional phosphate is removed using alkaline phosphatase or T₄ polynucleotide kinase. Suitable enzymes for removing the optional phosphate include, any phosphatase, for example, alkaline phosphatase such as shrimp alkaline phosphatase, bacterial alkaline phosphatase, or calf intestinal alkaline phosphatase.

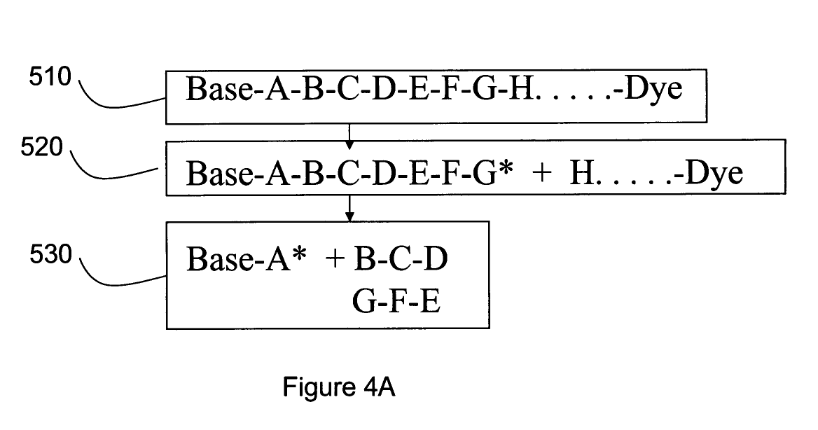

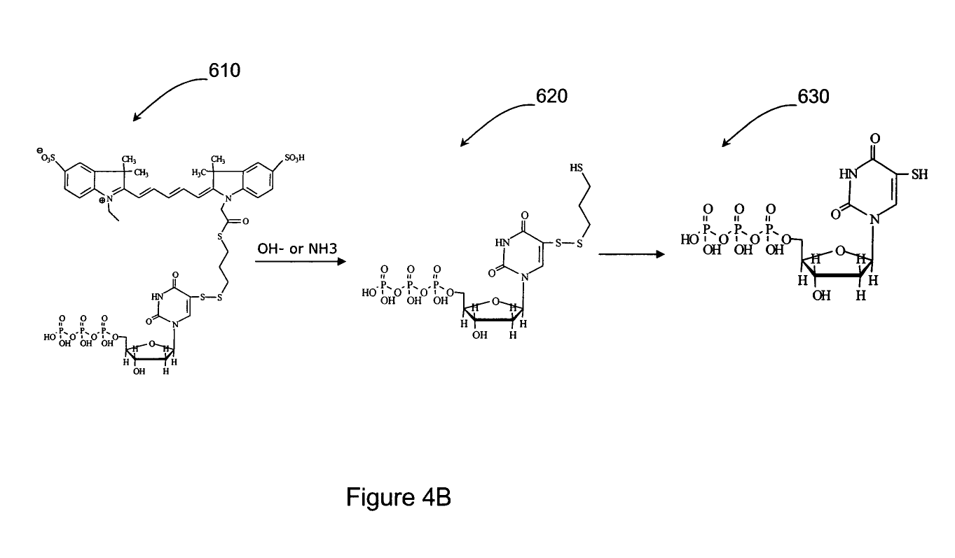

Referring now to FIGS. 3A and 3B. FIG. 3A shows a generic representation of a nucleotide analog of the invention having a linker A-B-C-D-E-F-G-H between the base and the label, a dye. A-H represent the number of atoms present in the backbone of the linker. The linker features a cleavable site. In order to minimize structural perturbation of the base following cleavage, the cleavable site is located close to the base. FIG. 3B shows exemplary chemical structures of nucleotide analogs of the invention having a linker between the base and the label. In FIG. 3B, one structure features a linker with a cleavage site 300 and the other structure features a linker with the cleavage site 400. The nucleotide analog having the cleavage site 300 is preferred relative to the nucleotide analog having the cleavage site 400, because after cleavage site 300 is cleaved the remaining portion of the linker has fewer atoms compared to the linker after cleavage site 400 is cleaved (although both structures are nucleotide analogs according to the invention). Initiation and control of cleavage may be by, for example, chemical means (e.g., initiation by adding one or more reactive chemical) or photochemical means (e.g., adding one or more forms of light).

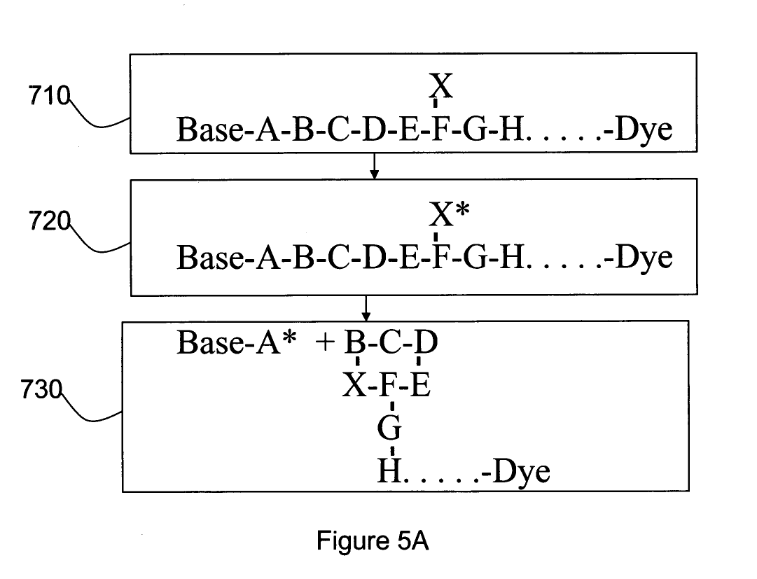

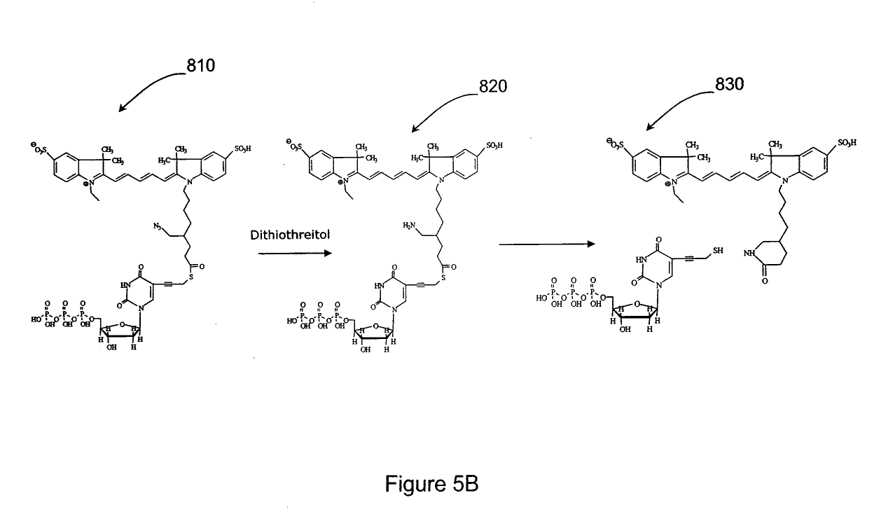

Referring now to FIGS. 4A and 4B. FIG. 4A shows a generic representation of nucleotide analogs of the present invention having a linker between the base and a label. Generally, the cleavage of the linker at a cleavage site removes a portion of the linker and a label. The portion of the cleaved linker that is attached to the base is reactive with another portion of the linker, thereby removing an additional portion of the linker. The remaining portion of the linker attached to the base is reactive. An exemplary mechanism for this reaction is shown in FIG. 4B. Specifically, referring still to FIG. 4A, a nucleotide analog has a linker A-B-C-D-E-F-G-H between the base and the label, a dye (Step 510). The linker features a cleavable site, for example, a cleavable bond between G-H that when cleaved removes the label and a portion of the linker (i.e., H) and produces G*, which is reactive and remains linked to the base (step 520). The cleaved site G* is reactive with B, G* and B react, and an additional portion of the linker (i.e, B-C-D-E-F-G) is removed from the base. The base maintains A*, which is itself reactive (step 530). Optionally, A* is capped, rendering it unreactive.

FIG. 4B shows exemplary chemical structures of nucleotide analogs of the present invention that first undergo cleavage to remove a portion of a linker and a label (steps 610 to 620) as described in FIG. 4A. Cleavage may take place in the presence of, for example, hydroxide or ammonia. Thereafter, the cleaved portion of the linker that remains attached to the base is reactive and undergoes internal rearrangement resulting in an additional section of the remaining linker being removed (steps 620 to 630). The linker remaining attached to the base may be reactive and can optionally be capped, rendering it unreactive.

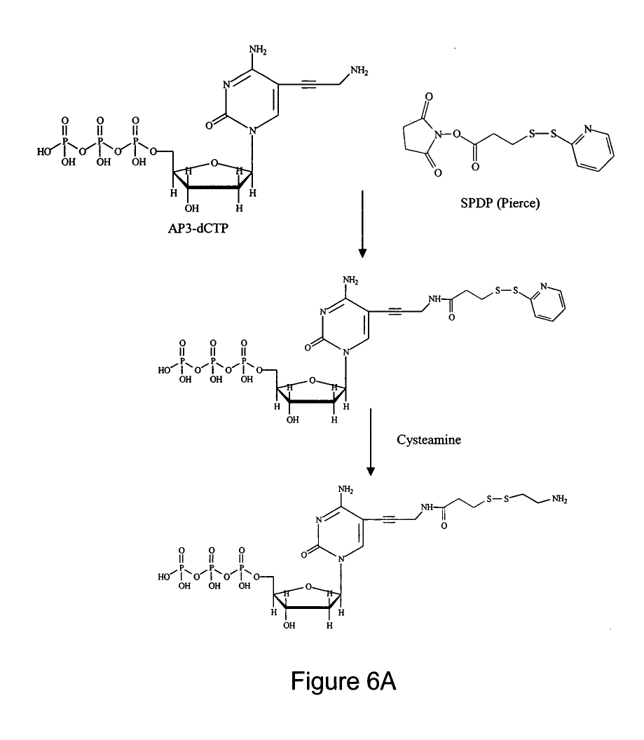

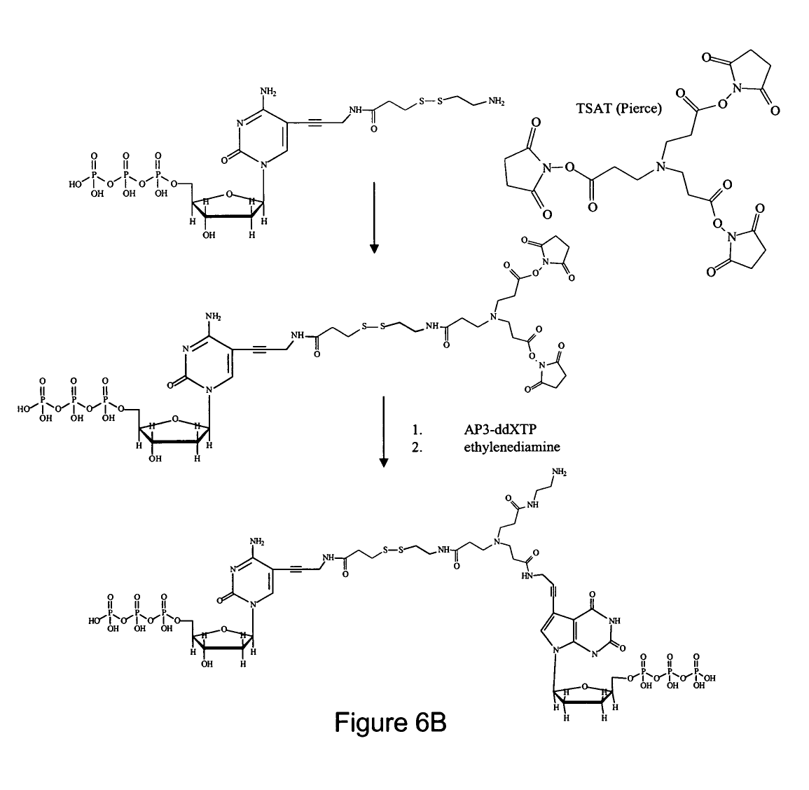

Referring now to FIGS. 5A and 5B, FIG. 5A shows a generic representation of nucleotide analogs of the invention that undergo cleavage of the linker between the base and a label. The nucleotide analog has a linker between the base and a label. The nucleotide analog initiates a cleavage event by activating an otherwise silent chemical entity that is attached to the linker. Activating the chemical entity by, for example, chemical means and/or photochemical means results in cleavage of the linker. Specifically, referring still to FIG. 5A, the linker features a silent chemical entity X and/or a silent chemical entity X is attached to the linker (step 710). The silent chemical entity X is exposed to an activation event (e.g., a light source), which activates the formerly silent chemical entity resulting in activation of the chemical entity X* (step 720). The activated chemical entity X* reacts with a chemical B within the linker. The reaction results in cleavage of bond A-B, removal of a portion of the linker and the dye, and the remaining portion of the linker, the cleaved bond A*, remains with the base (step 730).

FIG. 5B shows exemplary chemical structures of nucleotide analogs of the present invention that undergo cleavage of the linker between the base and a label as described in FIG. 5A. The nucleotide analog has a linker between the base and a label. The nucleotide analog initiates a cleavage event by activating an otherwise silent chemical entity that is attached to the linker. The silent chemical entity, N₃, is activated by chemical means, for example, exposure to Dithiothreitol, resulting in the activated chemical entity NH₂ (steps 810 and 820). Exposure to the activation event (i.e., Dithiothreitol) activates the formerly silent chemical entity N₃ resulting in NH₂ (steps 810 and 820). The activated chemical entity NH₂ reacts with the Sulfur-Carbon bond within the linker resulting in cleavage of the bond S—C on the linker and removal of a portion of the linker and the dye. The remaining portion of the linker, the cleaved Sulfur bond, bonds with a Hydrogen from the activated chemical entity and remains with the base (step 830).

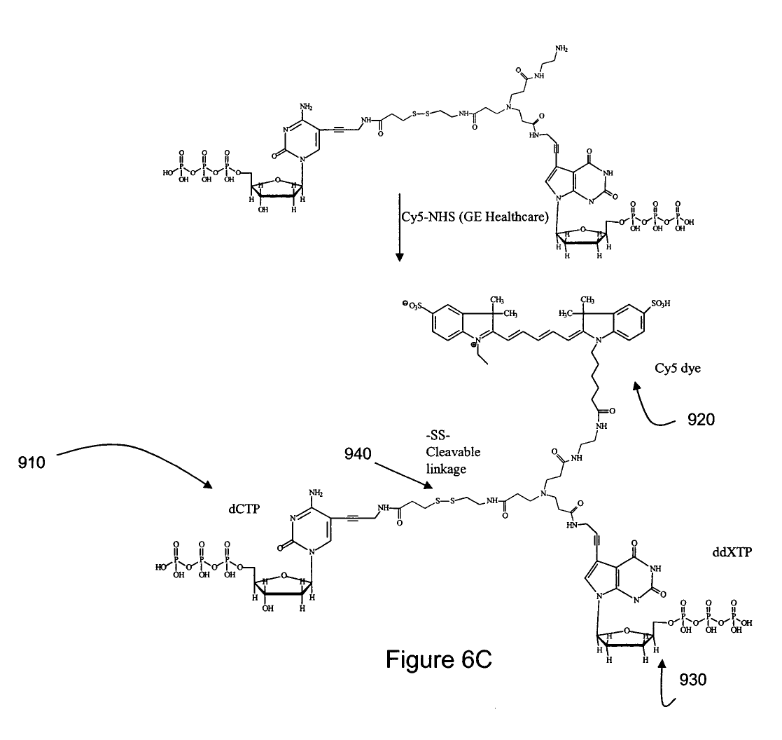

FIG. 6C shows another exemplary nucleotide analog having a cleavable linker. The nucleotide analog includes a dCTP attached to a dideoxynucleotide (ddXTP) by a cleavable linker. The nucleotide analog also is attached to a Cy5 label. The nucleotide analog 910 undergoes cleavage of the linker between the dCTP 910 and a label 920. The linker is cleaved by exposing the linker to one or more of chemical, photolytic, and/or mechanical cleavage processes. The linker is cleaved at the disulfide bond 940. Cleavage disassociates the Cy5 label 920, a portion of the cleavable linker, and a ddXTP 930 from the dCTP 910. The cleavable disulfide bond 940 of the linker is located close to the dCTP 910 so as to minimize structural perturbation of base, dCTP 910 following cleavage. After cleavage of the disulfide bond 940 fewer than ten atoms remain between the cleavage site 940 and the base, dCTP 910. FIGS. 6A-6C show an exemplary method of making a nucleotide analog discussed herein.

Although the dCTP 910 is depicted with a Cy5 dye as a label 920, as described herein, any suitable label that is compatible with the chemistry of the nucleotide analog and/or the linker may be employed. Additionally, the label 920 can be positioned in other locations within the nucleotide analog, for example, the label 920 can be bonded at the 3′ hydrogen position of the ddXTP base. The ddXTP base blocks further polymerization when added to the end of the primer (and/or template) by the polymerase. The ddXTP can be any appropriate base so long as the base lacks a 3′ hydroxyl group necessary for continued nucleic acid synthesis, such as, for example, a ddATP, ddCTP, ddGTP, ddTTP, or ddUTP.

Detection

Any detection method may be used to identify an incorporated nucleotide analog that is suitable for the type of label employed. Thus, exemplary detection methods include radioactive detection, optical absorbance detection, e.g., UV-visible absorbance detection, optical emission detection, e.g., fluorescence or chemiluminescence. Single-molecule fluorescence can be made using a conventional microscope equipped with total internal reflection (TIR) illumination. The detectable moiety associated with the extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T.G. Ed., Academic Press, Landon, pp. 1-11 (1993), such as described in Yershov et al., Proc. Natl. Aca. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc. Such detection methods are particularly useful to achieve simultaneous scanning of multiple attached target nucleic acids.

The present invention provides for detection of molecules from a single nucleotide to a single target nucleic acid molecule. A number of methods are available for this purpose. Methods for visualizing single molecules within nucleic acids labeled with an intercalating dye include, for example, fluorescence microscopy. For example, the fluorescent spectrum and lifetime of a single molecule excited-state can be measured. Standard detectors such as a photomultiplier tube or avalanche photodiode can be used. Full field imaging with a two-stage image intensified COD camera also can be used. Additionally, low noise cooled CCD can also be used to detect single fluorescent molecules.

The detection system for the signal may depend upon the labeling moiety used, which can be defined by the chemistry available. For optical signals, a combination of an optical fiber or charged couple device (CCD) can be used in the detection step. In those circumstances where the substrate is itself transparent to the radiation used, it is possible to have an incident light beam pass through the substrate with the detector located opposite the substrate from the target nucleic acid. For electromagnetic labeling moieties, various forms of spectroscopy systems can be used. Various physical orientations for the detection system are available and discussion of important design parameters is provided in the art.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Some embodiments of the present invention use TIRF microscopy for two-dimensional imaging. TIRF microscopy uses totally internally reflected excitation light and is well known in the art. See, e g., the World Wide Web at nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. The optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached target nucleic acid target molecule/primer complex in the presence of a polymerase. Total internal reflectance fluorescence microscopy is then used to visualize the attached target nucleic acid target molecule/primer complex and/or the incorporated nucleotides with single molecule resolution.

Fluorescence resonance energy transfer (FRET) can be used as a detection scheme. FRET in the context of sequencing is described generally in Braslavasky, et al., Proc. Nat'l Acad. Sci., 100: 3960-3964 (2003), incorporated by reference herein. Essentially, in one embodiment, a donor fluorophore is attached to the primer, polymerase, or template. Nucleotides added for incorporation into the primer comprise an acceptor fluorophore that is activated by the donor when the two are in proximity.

Measured signals can be analyzed manually or by appropriate computer methods to tabulate results. The substrates and reaction conditions can include appropriate controls for verifying the integrity of hybridization and extension conditions, and for providing standard curves for quantification, if desired. For example, a control nucleic acid can be added to the sample. The absence of the expected extension product is an indication that there is a defect with the sample or assay components requiring correction.

In one embodiment, the detectable moiety is attached to the pyrophosphate group, and the pyrophosphate group is removed from the nucleotide analog during primer extension. The pyrophosphate containing the detectable moiety can be removed from the template/primer duplexes into a detection all where the presence and/or amount of the detectable label is determined, for example, by excitation at a suitable wavelength and detecting the fluorescence.

### EXAMPLE

The 7249 nucleotide genome of the bacteriophage M13mp18 is sequenced using nucleotide analogs of the invention.

Purified, single-stranded viral M13mp18 genomic DNA is obtained from New England Biolabs. Approximately 25 ug of M13 DNA is digested to an average fragment size of 40 bp with 0.1 U Dnase I (New England Biolabs) for 10 minutes at 37° C. Digested DNA fragment sizes are estimated by running an aliquot of the digestion mixture on a precast denaturing (TBE-Urea) 10% polyacrylamide gel (Novagen) and staining with SYBR Gold (Invitrogen/Molecular Probes). The DNase I-digested genomic DNA is filtered through a YM10 ultrafiltration spin column (Millipore) to remove small digestion products less than about 30 nt. Approximately 20 pmol of the filtered DNase I digest was then polyadenylated with terminal transferase according to known methods (Roychoudhury, R and Wu, R. 1980, Terminal transferase-catalyzed addition of nucleotides to the 3′ termini of DNA. Methods Enzymol. 65(1):43-62.). The average dA tail length is about 50+/−5 nucleotides. Terminal transferase is then used to label the fragments with Cy3-dUTP. Fragments are then terminated with dideoxyTTP (also added using terminal transferase). The resulting fragments are again filtered with a YM10 ultrafiltration spin column to remove free nucleotides and stored in ddH₂O at −20° C.

Epoxide-coated glass slides are prepared for oligo attachment. Epoxide-functionalized 40 mm diameter #1.5 glass cover slips (slides) are obtained from Erie Scientific (Salem, N.H.). The slides are preconditioned by soaking in 3×SSC for 15 minutes at 37° C. Next, a 500 pM aliquot of 5′ aminated polydT(50) (polythymidine of 50 bp in length with a 5′ terminal amine) is incubated with each slide for 30 minutes at room temperature in a volume of 80 ml. The resulting slides have poly(dT50) primer attached by direct amine linker to the epoxide. The slides are then treated with phosphate (1 M) for 4 hours at room temperature in order to passivate the surface. Slides are then stored in polymerase rinse buffer (20 mM Tris, 100 mM NaCl, 0.001% Triton® X-100 (polyoxyethylene octyl phenyl ether), pH 8.0) until used for sequencing.

For sequencing, the slides are placed in a modified FCS2 flow cell (Bioptechs, Butler, Pa.) using a 50 um thick gasket. The flow cell is placed on a movable stage that is part of a high-efficiency fluorescence imaging system built around a Nikon TE-2000 inverted microscope equipped with a total internal reflection (TIR) objective. The slide is then rinsed with HEPES buffer with 100 mM NaCl and equilibrated to a temperature of 50° C. An aliquot of the M13 template fragments described above is diluted in 3×SSC to a final concentration of 1.2 nM. A 100 ul aliquot is placed in the flow cell and incubated on the slide for 15 minutes. After incubation, the flow cell is rinsed with 1×SSC/HEPES/0.1% SDS followed by HEPES/NaCl. A passive vacuum apparatus is used to pull fluid across the flow cell. The resulting slide contains M13 template/oligo(dT) primer duplex. The temperature of the flow cell is then reduced to 37° C. for sequencing and the objective is brought into contact with the flow cell.

For sequencing, cytosine triphosphate analog, guanidine triphosphate analog, adenine triphosphate analog, and uracil triphosphate analog, each having a fluorescent label, such as a Cy5, attached to the base via a cleavable linker (such as a disulfide bond). The cleavable link is located close to the base so as to minimize structural perturbation of base following cleavage. It is preferred that the cleavable linker includes between about 1 and 5 atoms between the cleavage site and the base as shown, for example, in FIGS. 3A and 3B. The analogs are stored separately in buffer containing 20 mM Tris-HCl, pH 8.8, 10 mM MgSO₄, 10 mM (NH₄)₂SO₄, 10 mM HCl, and 0.1% Triton® X-100 (polyoxyethylene octyl phenyl ether), and 100 U Klenow exo⁻ polymerase (NEN). Sequencing proceeds as follows.

First, initial imaging is used to determine the positions of duplex on the epoxide surface. The Cy3 label attached to the M13 templates is imaged by excitation using a laser tuned to 532 nm radiation (Verdi V-2 Laser, Coherent, Inc., Santa Clara, Calif.) in order to establish duplex position. For each slide only single fluorescent molecules imaged in this step are counted. Imaging of incorporated nucleotides as described below is accomplished by excitation of a cyanine-5 dye using a 635 nm radiation laser (Coherent). 5 uM of a Cy5-labeled CTP analog as described above is placed into the flow cell and exposed to the slide for 2 minutes. After incubation, the slide is rinsed in 1×SSC/15 mM HEPES/0.1% SDS/pH 7.0 (“SSC/HEPES/SDS”) (15 times in 60 ul volumes each, followed by 150 mM HEPES/150 mM NaCl/pH 7.0 (“HEPES/NaCl”) (10 times at 60 ul volumes)). An oxygen scavenger containing 30% acetonitrile and scavenger buffer (134 ul HEPES/NaCl, 24 ul 100 mM Trolox in MES, pH 6.1, 10 ul DABCO in MES, pH 6.1, 8 ul 2M glucose, 20 ul Nal (50 mM stock in water), and 4 ul glucose oxidase) is next added. The slide is then imaged (500 frames) for 0.2 seconds using an Inova301K laser (Coherent) at 647 nm, followed by green imaging with a Verdi V-2 laser (Coherent) at 532 nm for 2 seconds to confirm duplex position. The positions having detectable fluorescence are recorded. After imaging, the flow cell is rinsed 5 times each with SSC/HEPES/SDS (60 ul) and HEPES/NaCl (60 ul).

Next, the fluorescent label (e.g., the cyanine-5) is cleaved off of the incorporated CTP analogs. Initiation and control of cleavage may be accomplished by chemical or photochemical induction. For example, a reactive chemical or light can be added (as described above) to initiate the cleavage of the label from the analog. Where the cleavable linker is a disulfide bond, the Cy5 label is removed by introduction into the flow cell of 50 mM TCEP for 5 minutes, after which the flow cell was rinsed 5 times each with SSC/HEPES/SDS (60 ul) and HEPES/NaCl (60 ul), and the remaining nucleotide is capped with 50 mM iodoacetamide for 5 minutes followed by rinsing 5 times each with SSC/HEPES/SDS (60 ul) and HEPES/NaCl (60 ul). The scavenger is applied again in the manner described above, and the slide is again imaged to determine the effectiveness of the cleave/cap steps and to identify non-incorporated fluorescent objects.

In certain embodiments, as shown in FIGS. 4A and 4B, the cleavable linker can be followed by internal rearrangement so as to minimize structural perturbation of base following cleave. In other embodiments, as shown in FIGS. 5A and 5B, the initiation of the cleavage event can be accomplished via activation of a chemical entity attached to the cleavable linker that results in cleavage of a target linker that is preferably close to the base.

The procedure described above is then conducted 100 nM Cy5dATP analog, followed by 100 nM Cy5dGTP analog, and finally 500 nM Cy5dUTP, each as described above. The procedure (expose to nucleotide, polymerase, rinse, scavenger, image, rinse, cleave, rinse, cap, rinse, scavenger, final image, removal of optional phosphate group) is repeated exactly as described for ATP, GTP, and UTP except that Cy5dUTP is incubated for 5 minutes instead of 2 minutes. Uridine is used instead of thymidine due to the fact that the Cy5 label is incorporated at the position normally occupied by the methyl group in thymidine triphosphate, thus turning the dTTP into dUTP. In all 64 cycles (C, A, G, U) are conducted as described in this and the preceding paragraph.

Once 64 cycles are completed, the image stack data (i.e., the single molecule sequences obtained from the various surface-bound duplex) is aligned to the M13 reference sequence.

The alignment algorithm matches sequences obtained as described above with the actual M13 linear sequence. Placement of obtained sequence on M13 is based upon the best match between the obtained sequence and a portion of M13 of the same length, taking into consideration 0, 1, or 2 possible errors. All obtained 9-mers with 0 errors (meaning that they exactly matched a 9-mer in the M13 reference sequence) are first aligned with M13. Then 10-, 11-, and 12-mers with 0 or 1 error are aligned. Finally, all 13-mers or greater with 0, 1, or 2 errors are aligned.

All publications, patents, and patent applications cited herein are hereby expressly incorporated by reference in their entirety and for all purposes to the same extent as if each was so individually denoted. The patent application entitled “Nucleotide Analogs” filed on even date herewith (Attorney Docket Number: HEL-027) is expressly incorporated by reference.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is clamed is:

1. A nucleotide analog having the structure: 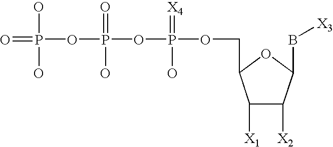
wherein X₁ is OH or PO₄;
X₂ is H or OH;
B is selected from the group consisting of a purine, a pyrimidine and derivatives thereof;
X₃ is a linker comprising between about 5 carbon atoms and about 1 carbon atom between a cleavable bond and the base B; and
X₄ is O or S.

2. The nucleotide analog of claim 1, wherein said linker X₃ is attached to a label.

3. The nucleotide analog of claim 2, wherein said label is an optically-detectable label.

4. The nucleotide analog of claim 3, wherein said optically-detectable label is a fluorescent label.

5. The nucleotide analog of claim 1, wherein B is selected from the group consisting of cytosine, uracil, thymine, adenine, guanine, and analogs thereof.

6. The nucleotide analog of claim 1, wherein X₃ comprises a cleavable bond.

7. The nucleotide analog of claim 6, wherein said cleavable bond X₃ is a chemically cleavable bond.

8. The nucleotide analog of claim 6, wherein said cleavable bond X₃ is a photochemically cleavable bond.

9. The nucleotide analog of claim 8, wherein said photochemically cleavable bond is selected from the group consisting of o-nitrobenzyl and derivatives thereof.

10. The nucleotide analog of claim 6, wherein said cleavable bond is selected from the group consisting of:

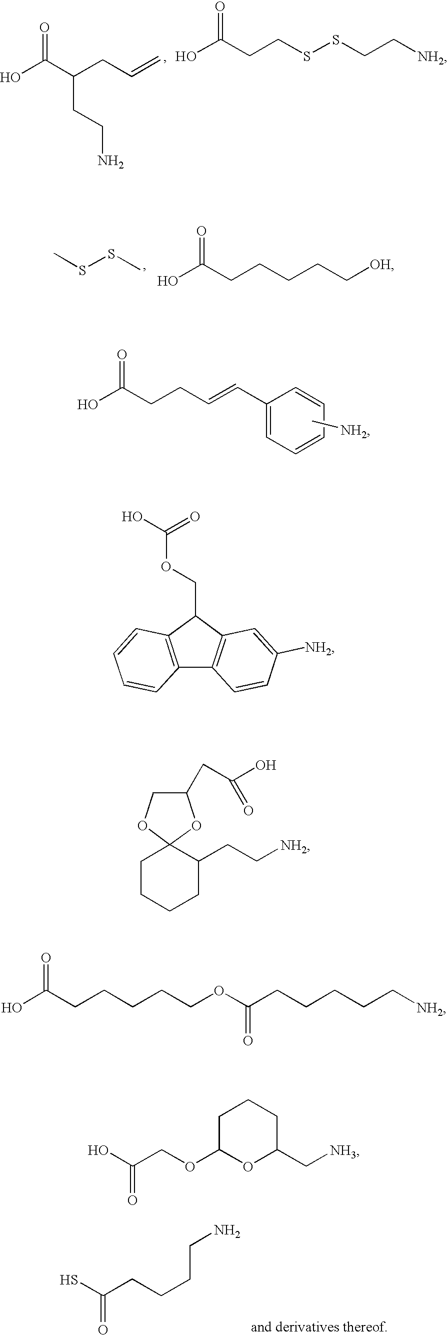

11. The nucleotide analog of claim 1, wherein X₃ comprises a triple bond.

12. The nucleotide analog of claim 1, wherein said linker X₃ has between 5 carbon atoms and 1 carbon atom.

13. A method for sequencing a nucleic acid template, the method comprising the steps of:
(a) exposing a nucleic acid template to a primer capable of hybridizing to said template and a polymerase capable of catalyzing nucleotide addition to said primer;
(b) incorporating a labeled nucleotide analog having a linker comprising between about 5 carbon atoms and about 1 carbon atom between a cleavable bond and a base;
(c) identifying said incorporated labeled nucleotide;
(d) cleaving said cleavable bond;
(e) repeating steps b, c, and d at least once; and
(f) determining a sequence of said template based upon the order of incorporation of said labeled nucleotides.

14. The method of claim 13 further comprising the step of:
(g) capping said cleavable bond.

15. The method of claim 13, wherein the label is a detectable label.

16. The method of claim 15, wherein said detectable label is selected from the group consisting of cyanine, rhodamine, fluorescien, coumarin, BODIPY, alexa, and conjugated multi-dyes.

17. The method of claim 13, wherein said template is individually optically resolvable.

18. The method of claim 13, wherein said template is attached to a surface.

19. The method of claim 13, wherein the polymerase is Klenow with reduced exonuclease activity.

20. A method for sequencing a nucleic acid template, the method comprising the steps of:
(a) exposing a nucleic acid template to a primer capable of hybridizing to said template and a polymerase capable of catalyzing nucleotide addition to said primer;
(b) incorporating a labeled nucleotide analog having a linker greater than 5 carbon atoms between a base and a label;
(c) identifying said incorporated labeled nucleotide;
(d) removing the label and at least a portion of said linker, wherein a remaining portion of said linker has 5 or fewer carbon atoms;
(e) repeating steps b, c, and d at least once; and
(f) determining a sequence of said template based upon the order of incorporation of said labeled nucleotides.

21. A method for sequencing a nucleic acid template, the method comprising the steps of:
(a) exposing a nucleic acid template to a primer capable of hybridizing to said template and a polymerase capable of catalyzing nucleotide addition to said primer;
(b) incorporating a labeled nucleotide analog having a linker comprising between about 5 carbon atoms and about 1 carbon atom;
(c) identifying said incorporated labeled nucleotide by a label;
(d) removing said label;
(e) repeating steps b, c, and d at least once; and
(f) determining a sequence of said template based upon the order of incorporation of said labeled nucleotides.
